(19) United States
(12) Patent Application Publication
(10) Pub. No.: US 2017/0266113 A1
(43) Pub. Date: Sep. 21, 2017

(54) NEBULIZABLE COMPOSITIONS OF QUATERNARY AMMONIUM MUSCARINIC RECEPTOR ANTAGONISTS

(71) Applicant: Mylan Speciality L.P., Basking Ridge, NJ (US)

(72) Inventor: Imtiaz Chaudry, American Canyon, CA (US)

(21) Appl. No.: 15/612,409

(22) Filed: Jun. 2, 2017

Related U.S. Application Data
(63) Continuation of application No. 13/629,194, filed on Sep. 27, 2012.
Application No. 13/629,194 is a division of application No. 12/704,767, filed on Feb. 12, 2010.
Application No. 12/704,767 is a continuation of application No. 11/753,648, filed on May 25, 2007, now abandoned.
(60) Provisional application No. 60/803,309, filed on May 26, 2006.

(51) Int. Cl. A61K 9/00

(52) U.S. Cl. CPC A61K 9/0078; A61K 9/08

(57) ABSTRACT

Compositions and methods for treatment, prevention, or amelioration of one or more symptoms of bronchoconstrictive disorders are provided. The compositions provided herein are nebulizable compositions comprising quaternary ammonium muscarinic receptor antagonists. The compositions are suitable for direct administration to a patient in need thereof via a nebulizer. Also provided are kits which comprise of the nebulizable composition of the invention in combination with a nebulizer. Also provided is a method of treating, preventing, or amelioration of one or more symptoms of bronchoconstrictive disorders by administering a therapeutically effective amount of the nebulizable composition of the invention via the use of a nebulizer to a patient in need thereof with minimal to no exposure of the nebulizable composition to the body surface of the patient.

This application claims the benefit of U.S. Provisional Patent Application Ser. No. 60/803,309, filed on May 26, 2006, the contents of which are incorporated by reference herein.

### FIELD OF THE INVENTION

Nebulizable compositions of quaternary ammonium muscarinic receptor antagonists and methods of using the nebulizable compositions for treatment, prevention, or amelioration of one or more symptoms of broncho-constrictive disorders to a patient in need thereof are provided. Also provided are kits containing the nebulizable composition in combination with a nebulizer for the delivery of the nebulizable composition to the lungs of a patient in need thereof with minimal to no exposure of the nebulizable composition to the body surface of the patient.

### BACKGROUND OF THE INVENTION

Bronchoconstrictive disorders affect millions worldwide. Such disorders include asthma (including bronchial asthma, allergic asthma and intrinsic asthma, e.g., late asthma and airway hyper-responsiveness), chronic bronchitis and other chronic obstructive pulmonary diseases.

Compounds for the treatment of bronchoconstrictive disorders are typically formulated for inhalation (aerosol) therapy. A problem associated with inhalation therapy is that about 90% of the active ingredient is swallowed and destroyed in the gastrointestinal tract and only about 10% of the active ingredient reaches the pulmonary tract. Exacerbating this problem is the difficulty of using inhalers to deliver the active ingredient. Studies have shown that more than 50% of patients using inhalers do not use the proper technique and thereby markedly reduce the amount of drug inhaled into the lungs while not reducing the amount deposited in the mouth. Goodman & Gilman's The Pharmacological Basis of Therapeutics (10^{th} Ed.—Intl Ed.), ed. Hardman et al., McGraw-Hill Med. Pub. Div., page 736, (2001); see also Epstein et al., “Survey of the clinical use of pressurized aerosol inhalers”, Can. Med. Assoc. J., 120: 813-816 (1979) and MacFarlane et al. “Irregularities in the use of regular aerosol inhalers”, Thorax, 35: 477-478 (1980).

One solution to this problem is to use dry powdered inhalers. However, the powdered compositions used in dry power inhalers are also difficult to administer, particularly to the young and elderly who are most often the patients in need of such therapy. In addition, powdered inhalers suffer from the problem of small amounts of powder being expelled into the air resulting in contact of the powdered active ingredient with the skin and/or eyes of the patient resulting in irritation to the patient and decreasing the amount of active ingredient delivered to the lungs.

As such, aqueous or liquid compositions are still preferred over solid compositions for inhalation therapy. However, delivery of aqueous or liquid composition in aerosol or nebulized form also produces the same problems as dry powder compositions.

Therefore, there is a need for nebulizable compositions for delivery of quaternary ammonium muscarinic receptor antagonists to a patient in need thereof which can be conveniently administered, does not result in irritation to the skin and/or eyes and delivers the active ingredient to the lungs in sufficient amounts to treat a broncho-constrictive disorder.

Citation or identification of any document in this application is not an admission that such document is available as prior art to the present invention.

### SUMMARY OF THE INVENTION

Compositions and methods for treatment, prevention, or amelioration of one or more symptoms of bronchoconstrictive disorders are provided. The compositions provided herein are nebulizable compositions comprising quaternary ammonium muscarinic receptor antagonists. The compositions are suitable for direct administration to a patient in need thereof via a nebulizer.

Also provided are kits which comprise of the nebulizable composition of the invention in combination with a nebulizer.

Also provided is a method of treating, preventing, or amelioration of one or more symptoms of bronchoconstrictive disorders by administering a therapeutically effective amount of the nebulizable composition of the invention via the use of a nebulizer to a patient in need thereof with minimal to no exposure of the nebulizable composition to the body surface of the patient.

Also provided is a method of treating, preventing, or amelioration of one or more symptoms of bronchoconstrictive disorders by administering once a day a therapeutically effective amount of the nebulizable composition of the invention via the use of a nebulizer to a patient in need thereof with minimal to no exposure of the nebulizable composition to the body surface of the patient.

More specifically, the present invention provides a kit for the treatment, prevention or amelioration or one or more symptoms of diseases or disorders associated with bronchoconstriction which comprises:

(i) a nebulizer;
(ii) a nebulizable composition for the treatment, prevention or amelioration or one or more symptoms of diseases or disorders associated with bronchoconstriction which comprises:

(a) a quaternary ammonium muscarinic receptor antagonist in a concentration based on the ammonium of between about 0.0005% and about 5% by weight;

(b) a pharmacologically acceptable fluid; and

(c) a pharmacologically acceptable preservative,

wherein the pH of the preparation is adjusted between about 2.0 to about 4.5 with an acid and the quaternary ammonium muscarinic receptor antagonist is dissolved in the fluid and optional includes pharmacologically acceptable complexing agent, stabilizer, a pharmacologically acceptable cosolvent, or other pharmacologically acceptable adjuvants and additives; and

(iii) packaging material which includes instructions for the administration of the

nebulizable composition to a patient in need of treatment, prevention or amelioration or one or more symptoms of diseases or disorders associated with bronchoconstriction; wherein the administration of the nebulizable composition by the nebulizer results in minimal exposure of the nebulized composition to the body surface of the patient.

The invention further provides for a method of treating, preventing or ameliorating one or more symptoms of diseases or disorders associated with bronchoconstriction which comprises of delivering the nebulizable composition via the nebulizer from the inventive kit, wherein the administration of the nebulizable composition by the nebulizer results in minimal exposure of the nebulized composition to the body surface of the patient.

The body surface of the patient, as noted by the inventive kit or inventive method, preferably includes the face and eyes.

Preferably, the loss of quaternary ammonium muscarinic receptor antagonists delivered to the mouth and lungs of the patient by the inventive kit or inventive method is less than 0.001% w/w.

The nebulizer of the inventive kit or inventive method releases the nebulized composition upon inhalation by the patient and ceases release of the nebulized composition when inhalation is stopped, e.g., the nebulizer is a breath actuated nebulizer.

Preferably, the quaternary ammonium muscarinic receptor antagonist of the inventive kit or inventive method is an ipratropium or tiotropium compound, e.g., tiotropium bromide.

Optionally, the nebulizable composition of the inventive kit or inventive method further comprises an additional compound for the treatment of bronchostriction which is selected from the group consisting of a β₂-adrenoreceptor agonist, a dopamine (D₂) receptor agonist, a steroidal anti-inflammatory agent, an anticholinergic agent, an IL-5 inhibitor, an antisense modulator of IL-5, a tryptase inhibitor, a leukotriene receptor antagonist, a 5-lapoxygenase inhibitor, an anti-IgE antibody, an antihistamine, an anti-allergic agent and mixtures thereof.

Preferably, the nebulizable composition of the inventive kit or inventive method is contained in a unit dose vial. In a further aspect, the instructions included with the inventive kit, or inventive method, recite administration of the nebulizable composition once a day prior to going to sleep.

Accordingly, it is an object of the invention to not encompass within the invention any previously known nebulizable composition of quaternary ammonium muscarinic receptor antagonists, process of making said composition or method of using said composition such that applicant(s) reserve the right and hereby disclose a disclaimer of any previously known compositions or method of using the composition.

### DEFINITIONS

Unless defined otherwise, all technical and scientific terms used herein have the same meaning as is commonly understood by one of ordinary skill in the art to which this invention belongs. All patents, applications, published applications and other publications are incorporated by reference in their entirety. In the event that there is a plurality of definitions for a term herein, those in this section prevail unless stated otherwise.

As used herein, a nebulized solution refers to a solution that is dispersed in air to form an aerosol. Thus, a nebulized solution is a particular form of an aerosol.

As used herein, a breath actuated nebulizer is an instrument that is capable of generating very fine liquid droplets for inhalation into the lung wherein the nebulizer is pressure sensitive so that the nebulization is coordinated with the breathing cycle of a patient. Within this instrument, the nebulizing liquid or solution is atomized into a mist of droplets with a broad size distribution by methods known to those of skill in the art, including, but not limited to, compressed air, ultrasonic waves, or a vibrating orifice. The nebulizers may further contain, e.g., a baffle which, along with the housing of the instrument, selectively removes large droplets from the mist by impaction. Thus, the mist inhaled into the lung contains fine aerosol droplets. In one embodiment of the breath actuated nebulizer, the nebulizer includes a relief piston to lower the inhalation effort required by the inhaling patient.

As used herein, a pharmacologically suitable fluid is a solvent suitable for pharmaceutical use which is not a liquified propellant gas. Exemplary pharmacologically suitable fluids include polar fluids, including protic fluids such as water.

As used herein, a combination refers to any association between two or among more items.

As used herein, fluid refers to any composition that can flow. Fluids thus encompass compositions that are in the form of semi-solids, pastes, solutions; aqueous mixtures, gels, lotions, creams and other such compositions.

As used herein, a mixture is a mutual incorporation of two or more substances, without chemical union, the physical characteristics of each of the components being retained.

As used herein, pharmaceutically acceptable derivatives of a compound include salts, esters, enol ethers, enol esters, acids, bases, solvates, hydrates or prodrugs thereof. Such derivatives may be readily prepared by those of skill in this art using known methods for such derivatization. The compounds produced may he administered to animals or humans without substantial toxic effects and are either pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates. lactates, malates, tartrates, citrates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl and heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl and heterocyclyl. Pharmaceutically acceptable solvates and hydrates are complexes of a compound with one or more solvent or water molecule selected from the range of 1 to about 100; 1 to about 10 and one to about 2, 3 or 4, solvent or water molecules.

As used herein, treatment means any manner in which one or more of the symptoms of a condition, disorder or disease are ameliorated or otherwise beneficially altered. Treatment also encompasses any known pharmaceutical use of quaternary ammonium muscarinic receptor antagonists.

As used herein, amelioration of the symptoms of a particular disorder by administration of a particular pharmaceutical composition refers to any lessening, whether permanent or temporary, lasting or transient that can be attributed to or associated with administration of the composition.

As used herein, a prodrug is a compound that, upon in vivo administration, is metabolized or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

It is to be understood that the compounds for use in the compositions and methods provided herein may contain chiral centers. Such chiral centers may be of either the (R) or (S) configuration, or may be a mixture thereof. Thus, the compounds for use in the compositions provided herein may be enantiomerically pure, or be stereoisomeric or diastereomeric mixtures. It is to be understood that the chiral centers of the compounds provided herein may undergo epimerization in vivo. Thus, one of skill in the art will recognize that administration of a compound in its (R) form is equivalent, for compounds that undergo epimerization in vivo, to administration of the compound in its (S) form.

As used herein, bronchoconstriction refers to a reduction in the caliber of a bronchus or bronchi.

As used herein, undesired and/or uncontrolled bronchoconstriction refers to bronchoconstriction that results in or from a pathological symptom or condition. Pathological conditions include, but are not limited to, asthma and chronic obstructive pulmonary disease (COPD). Pathological symptoms include, but are not limited to, asthma and COPD.

As used herein, conveniently administered refers to administration of a dosage amount of the nebulizable composition of the invention no more than twice a day. In another embodiment of the invention, the administration is only once per day.

It is noted that in this disclosure and particularly in the claims and/or paragraphs, terms such as “comprises”. “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments of the invention are disclosed or are apparent from and encompassed by, the following Detailed Description.

### DETAILED DESCRIPTION OF THE INVENTION

Surprisingly, the problems associated in the state of the art with regard to the administration of quaternary ammonium muscarinic receptor antagonists can be solved by the use of a breath actuated nebulizer to deliver the nebulizable composition. Although breath actuated nebulizers had been known in the art, their use was primarily focused on increasing the delivery of active agent to the lungs. Breath activated nebulizers deliver about double the amount (about 20-30% delivery rate) of active ingredient to the lungs compared to conventional aerosols or nebulizers, but still results in a significant amount of active ingredient that does not reach its intended target area, i.e. the lungs. However, despite the smaller particle size from use of the breath actuated nebulizer, the amount of active ingredient which makes contact with the skin and/or eyes is minimized. Thus, the efficacy of the treatment for bronchoconstrictive disorders is maintained while the side effects are minimized.

The nebulizable compositions of the invention include one or more quaternary ammonium muscarinic receptor antagonists or a pharmaceutically acceptable derivative thereof and a pharmacologically suitable fluid. Representative examples of suitable nebulizable compositions are those based upon the compositions described in U.S. Pat. No. 6,890,517, incorporated by reference herein in its entirety, which comprise:

(a) a first active substance comprising a quaternary ammonium muscarinic receptor antagonist in a concentration based on the ammonium of between about 0.0005% and about 5% by weight;
(a) a second active substance selected from the group consisting of an antiallergic, antihistamine, steroid and leukotriene antagonist;
(c) a pharmacologically acceptable fluid; and
(d) a pharmacologically acceptable preservative,
wherein the pH of the preparation is adjusted between about 2.0 to about 4.5 with an acid and the quaternary ammonium muscarinic receptor antagonist is dissolved in the fluid and optional includes pharmacologically acceptable complexing agent, stabilizer, a pharmacologically acceptable cosolvent, or other pharmacologically acceptable adjuvants and additives.

The pH of the formulation according to the invention are selected from the ranges consisting of between about 2.0 and about 4.5; between about 2.5 and about 3.5; between about 2.7 and about 3.5; between about 2.7 and about 3.2 and a pH with an upper limit of about 3.1.

The pH is adjusted by the addition of pharmacologically acceptable acids. Examples of inorganic acids which are an embodiment for this portion of the invention include: hydrochloric acid, hydrobromic acid, nitric acid, sulfuric acid, and/or phosphoric acid. Examples of other embodiments of organic acids are: ascorbic acid, citric acid, malic acid, tartaric acid, maleic acid, succinic acid, fumaric acid, acetic acid, formic acid, and/or propionic acid, etc. In one embodiment of this aspect of the invention, the inorganic acids are hydrochloric acid and sulfuric acid. It is also possible to use acids which form an acid addition salt with the active substance or, in the case of combined preparations, with one of the active substances.

Of the organic acids, ascorbic acid, fumaric acid and citric acid are one embodiment of this aspect of the invention, especially citric acid. If desired, mixtures of the abovementioned acids may also be used, particularly in the case of acids which have other properties in addition to their acidifying properties, e.g., those which act as flavorings or antioxidants, such as for example citric acid or ascorbic acid. Hydrochloric acid represents yet another embodiment of the inorganic acid.

If desired, pharmacologically acceptable bases may be used to precisely titrate the pH. Suitable bases include for example alkali metal hydroxides and alkali metal carbonates. The preferred alkali ion is sodium. If bases of this kind are used, care must be taken to ensure that the resulting salts, which are then contained in the finished pharmaceutical formulation, are pharmacologically compatible with the abovementioned acid.

According to the invention, there is no need to add edetic acid (EDTA) or one of the known salts thereof, e.g., sodium edetate, to the present formulation as a stabilizer or complexing agent.

Another embodiment of the invention, the nebulizable composition contains edetic acid and/or the salts thereof.

In a yet another embodiment with sodium edetate, the content based on sodium edetate is selected from a range consisting of less than about 10 mg/100 ml; from about 5 mg/100 ml to less than about 10 mg/100 ml and from greater than about 0 to about 5 mg/100 ml. In still another embodiment the content of sodium edetate is selected from a range of about 10 to about 30 mg/100 ml and not more than about 25 mg/100 ml.

In still another embodiment this additive is omitted entirely.

The remarks made concerning sodium edetate also apply analogously to other comparable additives which have complexing properties and can be used instead, such as for example nitrilotriacetic acid and the salts thereof.

By complexing agents is preferably meant within the scope of the present invention molecules which are capable of entering into complex bonds. Preferably, these compounds should have the effect of complexing cations, most preferably metal cations.

### A. Quaternary Ammonium Muscarinic Receptor Antagonists

Muscarinic receptor antagonists prevent the effects of acetylcholine by blocking its binding to muscarinic cholinergic receptors at neuroeffector sites on smooth muscle, cardiac muscle, and gland cells; in peripheral ganglia; and in the central nervous system. In general muscarinic receptor antagonists cause little blockade of the effects of acetylcholine at nicotinic receptor sites. However, quaternary ammonium analogs of atropine and related drugs generally exhibit a general degree of nicotinic blocking activity and, consequently, are more likely to interfere with ganglionic or neuromuscular transmission.

In the central nervous system (CNS), cholinergic transmission appears to be both muscarinic and nicotinic at spinal, subcortical, and cortical levels in the brain. At high or toxic doses, the central effects of atropine and related drugs generally consist of CNS stimulation followed by depression. Since quaternary compounds penetrate the blood-brain barrier poorly, antagonists of this type have little or no effects on the CNS. see Goodman & Gilman's The Pharmacological Basis of Therapeutics (10^{th} Ed.—Int'l. Ed.), ed. Hardman et al., McGraw-Hill Med. Pub. Div., page 162, (2001).

In one embodiment of the invention the quaternary ammonium muscarinic receptor antagonists include but are not limited to ipratropium, tiotropium, mixtures thereof and pharmaceutically acceptable derivatives thereof. The structures of the ipratropium and tiotropium ions are depicted in the structures below:

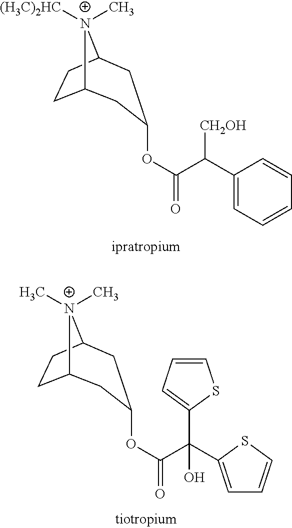

In another embodiment of the invention, the quaternary ammonium muscarinic receptor antagonist is a bromide of ipratropium or tiotropium. In yet another embodiment of the invention, the quaternary ammonium muscarinic receptor antagonist is a bromide of tiotropium.

In one embodiment of the invention, the pharmaceutically acceptable derivative is a pharmaceutically acceptable salt of the quaternary ammonium muscarinic receptor antagonist which include, but are not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. In one embodiment, the compositions for use in the methods provided herein contain formoterol fumarate or formoterol fumarate dihydrate. In another embodiment, the compositions for use in the methods provided herein contain formoterol tartrate.

In certain embodiments, the amount of quaternary ammonium muscarinic receptor antagonist, such as ipratropium bromide or tiotropium bromide, is in a concentration of about 5 μg/mL to about 5 mg/mL, or about 50 μg/mL to about 200 μg/mL. In other embodiments, the compositions for use in the methods herein contain an anticholinergic agent, including ipratropium bromide and tiotropium bromide, at a concentration of about 83 μg/mL to about 167 μg/mL.

### B. Other Agents for the Treatment of Bronchoconstrictive Disorders

In one embodiment of the nebulizable composition of the invention, the quaternary ammonium muscarinic receptor antagonist is combined with a β₂-adrenoreceptor agonist which includes but is not limited to Albuterol (α₁-(((1,1-dimethylethyl)amino)methyl)-4-hydroxy-1,3-benzenedimethanol); Bambuterol (dimethylcarbamic acid 5-(2-((1,1-dimethylethyl)amino)-1-hydroxyethyl)-1,3-phenylene ester); Bitolterol (4-methylbenzoic acid 4-(2((1,1-dimethylethyl)amino)-1-hydroxyethyl)-1,2-phenylene ester); Broxaterol (3-bromo-α-(((1,1-dimethylethyl)amino)methyl)-5-isoxazolemethanol); Isoproterenol (4-(1-hydroxy-2-((1-methylethyl)amino)ethyl)-1,2-benzenediol); Trimetoquinol (1,2,3,4-tetrahydro-1-((3,4,5-trimethoxyphenyl)methyl)-6,7-isoquinolinediol); Clenbuterol (4-amino-3,5-dichloro-α-(((1,1-diemthylethyl)amino)methyl)benzenemethanol); Fenoterol (5-(1-hydroxy-2-((2-(4-hydroxyphenyl)-1-methylethylamino)ethyl)-1,3-benzenediol); Formoterol (2-hydroxy-5-((1RS)-1-hydroxy-2-(((1RS)-2-(p-methoxyphenyl)-1-methylethyl) amino)ethyl)formanilide); (R,R)-Formoterol; Desformoterol ((R,R) or (S,S)-3-amino-4-hydroxy-α-(((2-(4-methoxyphenyl)-1-methylethyl)amino)methyl)benzenemethanol); Hexoprenaline (4,4′-(1,6-hexanediyl)-bis(imino(1-hydroxy-2,1-ethanediyl)))bis-1,2-benzenediol); Isoetharine (4-(1-hydroxy-2-((1-methylethyeamino)butyl)-1,2-benzenediol); Isoprenaline (4-(1-hydroxy-2-((methylethyl)amino)ethyl)-1,2-benzenediol); Metaproterenol (5-(1-hydroxy-2-((1-methylethyl)amino)ethyl)-1,3-benzenediol); Picumeterol (4-amino-3,5-dichloro-α-(((6-(2-(2-pyridinyl)ethoxy)hexyl)amino)methyl)benzenemethanol); Pirbuterol (α₆-(((1,1-dimethylethyl)amino)methyl)-3-hydroxy-2,6-pyridinemethanol); Procaterol (((R*,S*)-(±)-8-hydroxy-5-(1-hydroxy-2-((1-methylethyl)amino)butyl)-2(1 H)-quinolinone); Reproterol ((7-(3-((2-(3,5-dihydroxyphenyl)-2-hydroxyethyl)amino)propyl)-3,7-dihydro-1,3-dimethyl-1 H-purine-2,6-dione); Rimiterol (4-(hydroxy-2-piperidinylmethyl)-1,2-benzenediol); Salbutamol ((±)-α₁-(((1,1-dimethylethyl)amino)methyl)-4-hydroxy-1,3-benzenedimethanol); (R)-Salbutamol; Salmeterol ((±)-4-hydroxy-α₁-(((6-(4-phenylbutoxy)hexyl)amino)methyl)-1,3-benzenedimethanol); (R)-Salmeterol; Terbutaline (5-(2-((1,1-dimethylethyl)amino)-1-hydroxyethyl)-1,3-benzenediol); Tulobuterol (2-chloro-α-(((1,1-dimethylethyl)amino)methyl)benzenemethanol); and TA-2005 (8-hydroxy-5-((1R)-1-hydroxy-2-(N-((1R)-2-(4-methoxyphenyl)-1-methylethyl)amino)ethyl)carbostyril hydrochloride).

In one embodiment of the P₂ -adrenoreceptor agonist, the agonist is formoterol, or a pharmaceutically acceptable derivative thereof. In other embodiments, the formoterol for use in the compositions provided herein is formoterol fumarate. Formoterol refers to 2-hydroxy-5-((1RS)-1-hydroxy-2-(((1RS)-2-(p-methoxyphenyl)-1-methylethyl)amino)ethyl)formanilide; or a stereoisomer thereof. The term formoterol also refers herein to the single enantiomers 2-hydroxy-5-((1S)-1-hydroxy-2-(((1S)-2-(p-methoxyphenyl)-1-methylethyl)amino)ethyl)formanilide and 2-hydroxy-5-((1R)-1-hydroxy-2-4(1R)-2-(p-methoxyphenyl)-1-methylethyl)-amino)ethyl)formanilide.

In one embodiment of the use of formoterol, the nebulizable compositions contain formoterol free base at a concentration of about 5 μg/mL to about 2 mg/mL. In other embodiments, the maximum concentration of formoterol free base in the compositions is 1.5 mg/mL. In further embodiments, the concentration of fonnoterol free base in the compositions is about 10 μg/mL to about 1 mg/mL, or about 50 μg/mL to about 200 μg/mL.

In other embodiments, the compositions contain formoterol fumarate at a concentration of about 80 μg/mL up to about 175 to 200 μg/mL. In further embodiments, the compositions contain formoterol fumarate at a concentration of about 90 μg/mL up to about 125 to 150 μg/mL. The formoterol fumarate is formulated, in certain compositions provided herein, at a concentration of about 100 μg/mL. The formoterol fumarate is formulated. in other compositions provided herein; at a concentration of about 85 μg/mL or about 170 μg/mL. In one embodiment, the formoterol fumarate is formulated for single dosage administration via nebulization at a concentration of about 100 μg/mL. In another embodiment, the compositions contain formoterol free base at a concentration range of about 40 to about 150 μg/mL or about 59 to about 118 μg/mL.

Dopamine (D₂) receptor agonists may also be combined with the nebulizable composition of the invention and include, but are not limited to, Apomorphine ((r)-5,6,6a,7-tetrahydro-6-methyl-4H-dibenzo[de,g]quinoline-10,11-diol); Bromocriptine ((5′α)-2-bromo-12′-hydroxy-2′-(1-methylethyl)-5′-(2-methylpropyl)erg otaman-3′,6′,18-trione); Cabergoline ((8β)-N-(3(dimethylamino)propyl)-N-((ethylamino)carbonyl)6-(2-propenyl)ergoline-8-carboxamide); Lisuride (N′-((8α)-9,10-didehydro-6-methylergolin-8-yl)-N,N-diethylurea); Pergolide ((8β)-8-((methylthio)methyl)-6-propylergoline); Levodopa (3-hydroxy-L-tryrosine); Pramipexole ((s)-4,5,6,7-tetrahydro-N⁶-propyl-2,6-benzothiazolediamine); Quinpirole hydrochirodie (trans±)-4aR-4,4a,5,6,7,8,8a,9-octahydro-5-propyl-1H-pyrazolo[3,4-g]quinoline hydrochloride); Ropinirole (4-(2-(dipropylamino)ethyl)-1,3-dihydro-2H-indol-2-one); and Talipexole (5,6,7,8-tetrahydro-6-(2-propenyl)-4H-thiazolo[4,5-d]azepin-2-amine). Other dopamine D₂ receptor agonists for use herein are disclosed in International Patent Application Publication No. WO 99/36095.

Prophylactic therapeutics for use in combination therapy herein include steroidal anti-inflammatory agents, including, but not limited to, alclometasone, alclometasone dipropionate, alisactide, amcinonide, aminoglutethimide, aristocort diacetate, beclomethasone, beclomethasone-17,21-dipropionate, beclomethasone dipropionate (BDP), beclomethasone monopropionate (BMP), betamethasone valerate, betamethasone adamantoate, budesonide, butixocort, canesten-H C, ciclesonid, ciclometasone, clobetasol, clobetasone, cloprednol, cloprednol, fluocortin butyl, cortivazol, deflazacort, deflazacort, demetex, deprodone, deprodone propionate, dexamethasone, dexamethasone-21-isonicotinate, dexaniethasone isonicotinate, diflorasone, difluprednate, endrisone, fluazacort, fluclorolone acetonide, flunisolide, fluocinolone acetonide, fluocinonide, fluocortin, fluocortolone caproate, fluodexan, fluorometholone, fluticasone, fluticasone propionate, formebolone, formnocortal, halcinonide, halometasone, halopredone acetate, hydrocortisone, hydrocortisone-17-butyrate, hydrocortisone aceponate, hydrocortisone butyrate propionate, icomethasone enbutate, lotrisone, mazipredone, medrysone, meprednisone, methylprednisolone aceponate, mometasone, mometasone furoate, mycophenolate mofetil, pranlukast, paramethasone acetate, prednicarbate, promedrol, rofleponide, seratrodast, tipredan, tixocortol pivalate, triamcinolone, triamcinolone acetonide, triamcinolone hexacetonide, trilostane, triamcinolone benetonide, ulobetasol propionate, zileuton, and methyl 9-α-chloro-6-α-fluoro-11-β-17-α-dihydroxy-16-α-methyl-3-oxo-1,4-androstadiene-17-β-carboxylate-17-propionate, mometasone, mometasone furoate (Asmanex®, Twisthaler™, Shering-Plough Corporation, Kenilworth, N.J.), RPR 106541, sodium cromoglycate or nedocromil sodium.

Anticholinergic agents which may also be combined with the nebulizable composition of the invention for use herein include, but are not limited to, oxitropium bromide, atropine methyl nitrate, atropine sulfate, belladonna extract, scopolamine, scopolamine methobromide, homatropine methobromide, hyoscyamine, isopriopramide, orphenadrine, benzalkonium chloride and glycopyrronium bromide. Other active ingredients for use herein in combination therapy, include, but are not limited to, IL-5 inhibitors such as those disclosed in U.S. Pat. Nos. 5,668,110, 5,683,983, 5,677,280 and 5,654,276; antisense modulators of IL-5 such as those disclosed in U.S. Pat. No. 6,136,603; milrinone (1,6-dihydro-2-methyl-6-oxo-[3,4′-bipyridine]-5-carbonitrile); milrinone lactate; tryptase inhibitors such as those disclosed in U.S. Pat. No. 5,525,623; tachykinin receptor antagonists such as those disclosed in U.S. Pat. Nos. 5,691,336, 5,877,191, 5,929,094, 5,750,549 and 5,780,467; leukotriene receptor antagonists such as montelukast sodium (Singular®, R-(E)]-1-[[[1-[3-[2-(7-chloro-2-quinolinyl)ethenyl]phenyl]-3-[2-(1-hydroxy-1-methylethyl)phenyl]propyl]thio]methyl]cyclopropaneacetic acid, monosodium salt), 5-lapoxygenase inhibitors such as zileuton (Zyflo®, Abbott Laboratories, Abbott Park, Ill.), and anti-IgE antibodies such as Xolair® (recombinant humanized anti-IgE monoclonal antibody (CGP 51901; IGE 025A; rhuMAb-E25), Genentech, Inc., South San Francisco, Calif.), montelukast, pranlukast, zafirlukast, 1-(((R)-(3-(2-(6,7-difluoro-2-quinolinyl)ethenyl)phenyl)-3-(2-(2-hydroxy-2-propyl)phenyl)thio)methylcyclopropane acetic acid, 1-(((R)-3-(3-(2-(2,3-dichlorothieno[3,2-b]pyridin-5-yl)-(E)-ethenyl)phenyl)-3-(2-(1-hydroxy-1-methylethyl)phenyl)propyl)thio)methyl)cyclopropane acetic acid or [2-[[2-(4-tert-butyl-2-thiazolyl)-5-benzofuranyl]oxymethyl]phenyl]acetic acid. Examples of antihistamines and antiallergic agents: azelastine, astemizole, bamipine, carbinoxamine hydrogen maleate, cetirizine, dexchlorpheniramine, chlorphenoxamine, clemastine, clemastine hydrogen fumarate, desloratadine, dimenhydrinate, dimethindene, disodium cromoglycate, diphenhydramine, doxylamine, ebastine, emedastine, epinastinc, fexofenadine, ketotifen, levocabastine, loratadine, meclozine, mequitazine, mizolastine, nedocromil, pheniramine, and promethazine.

### C. Pharmaceutically Acceptable Fluids

The nebulizable compositions containing the quaternary ammonium muscarinic receptor antagonist, such as ipratropium or tiotropium, are formulated with a pharmacologically suitable fluid for the dissolution of the antagonist to facilitate nebulization and delivery of the antagonist into the lungs of a patient. Pharmacologically suitable fluids include, but are not limited to, polar solvents, including, but not limited to, compounds that contain hydroxyl groups or other polar groups. Such solvents include, but are not limited to, water or alcohols, such as ethanol, isopropanol, and glycols including propylene glycol, polyethylene glycol, polypropylene glycol, glycol ether, glycerol and polyoxyethylene alcohols.

Polar solvents also include protic solvents, including, but not limited to, water, aqueous saline solutions with one or more pharmaceutically acceptable salt(s), alcohols, glycols or a mixture thereof. For a saline solution as the solvent or as a component othereof, particularly suitable salts are those which display no or only negligible pharmacological activity after administration.

In another embodiment for the pharmaceutically acceptable fluid, the nebulizable compositions further contain a buffer, including, but not limited to, citric acid/phosphate, acetate, barbital, borate, Britton-Robinson, cacodylate, citrate, collidine, formate, maleate, Mcllvaine, phosphate, Prideaux-Ward, succinate, citrate-phosphate-borate (Teorell-Stanhagen), veronal acetate, MES (2-(N-morpholino)ethanesulfonic acid), BIS-TRIS (bis(2-hydroxyethyl)iminotris(hydroxymethyl)methane), ADA (N-(2-acetamido)-2-iminodiacetic acid), ACES (N-(carbamoylmethyl)-2-aminoethanesulfonaic acid), PIPES (piperazine-N,N′-bis(2-ethanesulfonic acid)), MOPSO (3-(N-morpholino)-2-hydroxypropanesulfonic acid), BIS-TRIS PROPANE (1,3-bis(tris(hydroxymethyl)methylamino)propane), BES (N,N-bis(2-hydroxyethyl)-2-aminoethanesulfonaic acid), MOPS (3-(N-morpholino)propanesulfonic acid), TES (N-tris(hydroxymethypmethyl-2-aminoethanesulfonic acid), HEPES (N-(2-hydroxyethyl)piperazine-N′-(2-ethanesulfonic acid), DIPSO (3-(N,N-bis(2-hydroxyethypamino)-2-hydroxypropanesulfonic acid), MOBS (4-(N-morpholino)butanesulfonic acid), TAPSO (3-(N-tris(hydroxymethyl)methylamino)-2-hydroxypropanesulfonic acid), TRIZMA® (tris(hydroxymethylaminomethane), HEPPSO (N-(2-hydroxyethyDpiperazine-N′-(2-hydroxypropanesulfonic acid), POPSO (piperazine-N,N′-bis(2-hydroxypropanesulfonic acid)), TEA (triethanolamine), EPPS (N-(2-hydroxyethyl)piperazine-N′-(3-propanesulfonic acid), TRICINE (N-tris(hydroxymethyl)methylglycine), GLY-GLY (glycylglycine), BICINE (N,N-bis(2-hydroxyethyl)glycine), HEPBS (N-(2-hydroxyethyl)piperazine-N′-(4-butanesulfonic acid)), TAPS (N-tris(hydroxymethyl)methyl-3-aminopropanesulfonic acid), AMPD (2-amino-2-methyl-1,3-propanediol), and/or any other buffers known to those of skill in the art. In one embodiment, the buffer is citric acid/phosphate buffer, acetate buffer, citrate buffer or phosphate buffer. In another embodiment, the buffer is a citrate buffer (citric acid/sodium citrate). The buffer concentration has been found herein to affect the stability of the composition. Buffer concentrations for use herein include from about 0 or 0.01 mM to about 150 mM, or about 1 mM to about 20 mM. In one embodiment, the buffer concentration is about 5 mM. In another embodiment, the buffer concentration is about 1 mM to about 50 mM, or about 20 mM.

In embodiments where the pharmacologically suitable fluid is a saline solution, tonicity adjusting agents may be added to provide the desired ionic strength. Tonicity adjusting agents for use herein include those which display no or only negligible pharmacological activity after administration. Both inorganic and organic tonicity adjusting agents may be used in the compositions provided herein. Tonicity adjusting agents include, but are not limited to, ammonium carbonate, ammonium chloride, ammonium lactate, ammonium nitrate, ammonium phosphate, ammonium sulfate, ascorbic acid, bismuth sodium tartrate, boric acid, calcium chloride, calcium disodium edetate, calcium gluconate, calcium lactate, citric acid, dextrose, diethanolamine, dim ethylsulfoxide, edetate disodium, edetate trisodium monohydrate, fluorescein sodium, fructose, galactose, glycerin, lactic acid, lactose, magnesium chloride, magnesium sulfate, mannitol, polyethylene glycol, potassium acetate, potassium chlorate, potassium chloride, potassium iodide, potassium nitrate, potassium phosphate, potassium sulfate, propylene glycol, silver nitrate, sodium acetate, sodium . bicarbonate, sodium biphosphate, sodium bisulfite, sodium borate, sodium bromide, sodium cacodylate, sodium carbonate, sodium chloride, sodium citrate, sodium iodide, sodium lactate, sodium metabisulfite, sodium nitrate, sodium nitrite, sodium phosphate, sodium propionate, sodium succinate, sodium sulfate, sodium sulfite, sodium tartrate, sodium thiosulfate, sorbitol, sucrose, tartaric acid, triethanolamine, urea, urethan, uridine and zinc sulfate. In certain embodiments, the tonicity adjusting agent is sodium chloride, which is present at a concentration of from about 0 mg/mL to about 10, 15 or 20 mg/mL. In further embodiments, the compositions contain sodium chloride at a concentration of from about 0 mg/mL to about 7.5 mg/mL. In another embodiment, the compositions contain sodium chloride at a concentration of 0 mg/mL, 1.5 mg/mL, 6.8 mg/mL or 7.5 mg/mL. In these embodiments, the pharmacologically suitable fluid is aqueous saline.

The nebulizable compositions provided herein also may include excipients and additives such as those described in Remington—The Science and Practice of Pharmacy (21^{st} Edition) (2005), Goodman & Gilman's The Pharmacological Basis of Therapeutics (11^{th} Edition) (2005) and Ansel's Pharmaceutical Dosage Forms and Drug Delivery Systems (8^{th} Edition), edited by Allen et al., Lippincott Williams & Wilkins, (2005). The particular excipient or additive for use in the nebulizable compositions provided herein may be determined empirically using methods well known to those of skill in the art (see, e.g., the Examples). Excipients and additives are any pharmacologically suitable and therapeutically useful substance which is not an active substance. Excipients and additives generally have no pharmacological activity, or at least no undesirable pharmacological activity. The excipients and additives include, but are not limited to, surfactants, stabilizers, complexing agents, antioxidants, or preservatives which prolong the duration of use of the finished pharmaceutical composition, flavorings, vitamins, or other additives known in the art. Complexing agents include, but are not limited to, ethylenediaminetetraacetic acid (EDTA) or a salt thereof, such as the disodium salt, citric acid, nitrilotriacetic acid and the salts thereof. In one embodiment, the complexing agent is EDTA. Preservatives include, but are not limited to, those that protect the solution from contamination with pathogenic particles, including benzalkonium chloride or benzoic acid, or benzoates such as sodium benzoate. Antioxidants include, but are not limited to, vitamins, provitamins, ascorbic acid, vitamin E or salts or esters thereof.

The compositions provided herein also may include a cosolvent, which increases the solubility of additives or the active ingredient(s). The particular cosolvent for use in the compositions for long term storage provided herein may be determined empirically using methods well known to those of skill in the art (see, e.g., the Examples). Cosolvents for use herein include, but are not limited to, hydroxylated solvents or other polar solvents, such as alcohols such as isopropyl alcohol, glycols such as propylene glycol, polyethylene glycol, polypropylene glycol, glycol ether, glycerol, and polyoxyethylene alcohols.

### D. Preparation of Compounds for Use in the Compositions

The preparation of the compounds and pharmaceutically acceptable derivatives thereof used in the compositions provided herein is described below. Any such compound or similar compound may be synthesized according to a method discussed in general below or by only minor modification of the methods by selecting appropriate starting materials. For example, the preparation of ipratropium compounds are described in U.S. Pat. No. 3,505,337 and the preparation of tiotropium compounds are described in U.S. Pat. No. 5,610,163 (equivalent to EP 418 716 and JP 7030071B), incorporated by reference herein. Each reference generally describes the derivatization of a tropine to produce the respective ipratropium or tiotropium compound.

### E. Preparation of Nebulizable Compositions

The compositions provided herein are prepared by procedures well known to those of skill in the art which include but are not limited to the procedures generally described in Remington—The Science and Practice of Pharmacy (21^{st} Edition) (2005), Goodman & Gilman's The Pharmacological Basis of Therapeutics (11^{th} Edition) (2005) and Ansel's Pharmaceutical Dosage Forms and Drug Delivery Systems (8^{th} Edition). For example, a tiotropium bromide solution may be prepared by the procedure of EXAMPLE 1.

### F. Evaluation of the Activity of the Compositions

Standard physiological, pharmacological and biochemical procedures are available for testing the compositions provided herein to identify those that possess bronchodilatory activity.

In vitro and in vivo assays that may be used to evaluate bronchodilatory activity are well known to those of skill in the art. See also, e.g., U.S. Pat. Nos. 3,994,974, and 6,068,833; German Patent No. 2,305,092; Kaumann et al. (1985) Naunyn-Schmied Arch. Pharmacol. 331:27-39; Lemoine et al. (1985) Naunyn-Schmied Arch. Pharmacol. 331:40-51; Tomioka et al (1981) Arch. Int. Pharmacodyn. 250:279-292; Dellamary et al. (2000) Pharm. Res. 17(2):168-174; Rico-Mendez et al. (1999) Rev. Alerg. Mex. 46(5):130-135; Seberova et al. (2000) Respir. Med. 94(6):607-61 I ; Lotvall et al. (1999) Can. Respir. J. 6(5):412-416; Campbell et al. (1999) Respir. Med. 93(4):236-244; Nightingale et al. (1999) Am. J. Respir. Crit. Care Med. 159(6): 1786-1790; Lecaillon et al. (1999) Eur. J. Clin. Pharmacol. 55(2): 131-138; Bartow et al. (1998) Drugs 55(2):303-322; Ekstrom et al. (1998) Respir. Med: 92(8):1040-1045; Ringdal et al. (1998) Respir. Med. 92(8):1017-1021; Totterman et al. (1998) Eur. Respir. J. 12(3):573-579; Palmqvist et al. (1997) Eur. Respir. J. 10(11):2484-2489; Nielsen et al. (1997) Eur. Respir. J. 10(9):2105-2109; Ullman et al. (1996) Allergy 51(10):745-748; Selroos et al. (1996) Clin. Immunother. 6:273-299; and Schreurs et al. (1996) Eur. Respir. J. 9(8):1678-1683.

### G. Nebulizers

Nebulizers suitable for use in the invention are those which minimize exposure of the nebulized composition to the body surface of the treated patient. In one embodiment of the invention, the nebulizer minimizes exposure of the nebulized composition to the face and eyes of the treated patient.

In one embodiment of the invention, the nebulizable compositions provided herein are intended for administration to a subject in need of such treatment via a breath actuated nebulizer. In one embodiment of the breath actuated nebulizer, the nebulizer is selected from the group consisting of AeroEclipse Breath Actuated Nebulizer and Autohaler®. AeroEclipse is described in U.S. Pat. Nos. 5,823,179 and 6,044,841 both of which are incorporated by reference herein in their entireties.

Simply by way of example, the U.S. Pat. No. 5,823,179 describes a nebulizer that includes:

a housing having a chamber for holding an aerosol;

a chamber air outlet communicating with said chamber for permitting said aerosol to he withdrawn from the chamber;

a liquid outlet located in the chamber;

a pressurized gas outlet located in the chamber adjacent to the liquid outlet; and

a movable diverter located in the chamber and spaced from the pressurized gas outlet and the liquid outlet by a variable height nebulizing gap, wherein the movable diverter is movable between a nebulizing position and a non-nebulizing position so as to divert pressurized gas from the gas outlet across the liquid outlet to produce the aerosol in cycles in response to a patient's breathing.

Simply by way of another example, U.S. Pat. No. 6,044,84 describes a nebulizer that includes:

a housing having a chamber for holding an aerosol;

an air outlet communicating with the chamber for permitting the aerosol to be withdrawn from the chamber;

a liquid orifice in communication with the chamber;

a pressurized gas inlet adjacent the liquid orifice, the pressurized gas inlet in communication with the chamber;

a diverter movably positioned in the chamber and relative to the pressurized gas inlet and the liquid orifice so as to divert pressurized gas from the pressurized gas inlet and over the liquid orifice when the diverter is in a nebulizing position; and,

a valve assembly comprising:

an actuator piston connected to the diverter and positioned in the chamber, the actuator piston responsive to an initial period of inhalation through the air outlet to move the diverter into the nebulizing position; and

a relief piston located in the chamber, the relief piston movable relative to the housing, independently moveable relative to the actuator piston, and responsive to additional negative pressure in the chamber after the initial period of inhalation to allow increased air flow into the chamber, whereby the effort necessary for a patient inhaling through the air outlet is maintained in a desired range.

In another embodiment of the invention, the nebulizer is any other device which operates under the principles of breath actuation, i.e. inhalation by the patient releases the nebulizable composition from the nebulizer into the mouth or nose and cessation of inhalation stops the release of the nebulizable composition.

In yet another embodiment of the invention, the nebulizer is used in conjunction with a mask which isolates the body surface such as the face and eyes from any escaping nebulized composition.

### H. Articles of Manufacture (Kits)

The nebulizable compositions provided herein may be packaged as articles of manufacture (a kit) containing packaging material, a composition provided herein, which is usefiil for treatment, prevention or amelioration of one or more symptoms of diseases or disorders associated with undesired and/or uncontrolled bronchoconstriction, a nebulizer and a label that indicates that the composition is used for treatment, prevention or amelioration of one or more symptoms of diseases or disorders associated with undesired and/or uncontrolled bronchoconstriction.

The nebulizable compositions are sterile filtered and filled in vials, including unit dose vials providing sterile unit dose compositions which are used in a nebulizer and suitably nebulized. Each unit dose vial is sterile and is suitably nebulized without contaminating other vials or the next dose. In one embodiment of the invention, a kit may contain one or more unit dosages of the nebulizable composition of the invention.

The unit dose vials are formed in a form-fill-seal machine or by any other suitable method known to those of skill in the art. The vials may be made of plastic materials that are suitably used in these processes. For example, plastic materials for preparing the unit dose vials include, but are not limited to, low density polyethylene, high density polyethylene, polypropylene and polyesters. In one embodiment, the plastic material is low density polyethylene.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected composition and intended mode of administration and treatment.

In one embodiment herein, the nebulizable compositions are packaged with a breath actuated nebulizer for direct administration of the composition to a subject in need thereof.

### I. Methods of Treatment of Bronchoconstrictive Disorders

The nebulizable compositions provided herein are used for treating, preventing, or ameliorating one or more symptoms of a bronchoconstrictive disorders in a subject. In one embodiment, the method includes administering to a subject an effective amount of a nebulizable composition containing a quaternary ammonium muscarinic receptor antagonist via a nebulizer, whereby the disease or disorder is treated or prevented without exposure of the nebulizable composition to the body surface of the patient.

In another embodiment of the treatment method, the body surface is the face and eyes.

In another embodiment of the treatment method, the nebulizer is a breath actuated nebulizer.

In another embodiment of the invention the quaternary ammonium muscarinic receptor antagonist is a bromide of ipratropium or tiotropium. In yet another embodiment of the invention, the quaternary ammonium muscarinic receptor antagonist is a bromide of tiotropium.

The methods for treatment, prevention, or amelioration of one or more symptoms of bronchoconstrictive disorders, in another embodiment, further include administering one or more of (a). (b). (c) or (d) as follows: (a) a β₂-adrenoreceptor agonist; (b) a dopamine (D₂) receptor agonist; (c) a prophylactic therapeutic, such as a steroid; or (d) an anticholinergic agent; simultaneously with, prior to or subsequent to the composition provided herein.

The subject treated is, in certain embodiments, a mammal. The mammal treated is, in certain embodiments, a human.

In another embodiment of the invention, the method provided herein reduces or eliminates the exposure of the body surface of a patient undergoing treatment to the nebulized composition. In one embodiment for the reduction of irritation, is achieved by no loss of active ingredient to the atmosphere which refers to less than 0.001% w/w loss of quaternary ammonium muscarinic receptor antagonists to delivery to the mouth or lungs. In another embodiment of the invention, no loss of active ingredient refers to less than 0.0001% w/w loss of quaternary ammonium muscarinic receptor antagonists to delivery to the mouth or lungs. In another embodiment of the invention, no loss of active ingredient refers to less than 0,00001% w/w loss of quaternary ammonium muscarinic receptor antagonists to delivery to the mouth or lungs.

In another embodiment of the invention, the method provided herein reduces the exposure of the face and/or eyes to a patient undergoing treatment by administering the nebulizable composition via a breath actuated nebulizer once a day. In another embodiment of the reduction of reducing exposure, the administration is performed prior to the patient going to sleep.

The bronchoconstrictive disorder to be treated, prevented, or whose one or more symptoms are to be ameliorated is associated with asthma, including, but not limited to, bronchial asthma, allergic asthma and intrinsic asthma, e.g., late asthma and airway hyper-responsiveness; and, particularly in embodiments where an anticholinergic agent is used, other chronic obstructive pulmonary diseases (COPDs), including, but not limited to, chronic bronchitis, emphysema, and associated cor pulmonale (heart disease secondary to disease of the lungs and respiratory system) with pulmonary hypertension, right ventricular hypertrophy and right heart failure. COPD is frequently associated with cigarette smoking, infections, environmental pollution and occupational dust exposure.

The following examples are included for illustrative purposes only and are not intended to limit the scope of the invention.

### EXAMPLE 1

### Preparation of the Quaternary Ammonium Muscarinic Receptor Antagonist Containing Nebulizable Composition

Nebulizable compositions of the invention may include compositions with the following ingredients and amounts:

| Ingredient | Amount |
|---|---|
| Tiotropium bromide | 5 μg-5 mg |
| Preservative | 5-15 mg |
| Buffer | 0-30 mg |
| HCl (1N) | ad pH 2.5-4.0 |
| Water | q.s. 100 mL |

### EXAMPLE 2

### Administration of the Nebulizable Composition

The nebulizable composition of Example I may be sterilized and inserted in a unit dose vial which is then inserted into a breath actuated nebulizer. The patient breathes into the nebulizer (which optionally contains a mask covering the nose and mouth) to deliver the unit dosage into the lungs. Administration of the unit dose is conducted prior to the patient sleeping to minimize the adverse affects of tiotropium bromide exposed to the atmosphere. It is expected that less than 0.001% w/w loss of tiotropium bromide occurs when adminstered with a breath actuated nebulizer.

Having thus described in detail various embodiments of the present invention, it is to he understood that the invention defined by the above paragraphs is not to be limited to particular details set forth in the above description as many apparent variations thereof are possible without departing from the spirit or scope of the present invention.

Any foregoing applications, and all documents cited therein or during their prosecution (“application cited documents”) and all documents cited or referenced in the application cited documents, and all documents cited or referenced herein (“herein cited documents”). and all documents cited or referenced in herein cited documents, together with any manufacturer's instructions, descriptions, product specifications, and product sheets for any products mentioned herein or in any document incorporated by reference herein, are hereby incorporated herein by reference herein, and may be employed in the practice of the invention.

1. A kit for the treatment or amelioration of one or more symptoms of disorders associated with bronchoconstriction which comprises:
(i) a nebulizer;
(ii) a nebulizable composition for the treatment or amelioration of one or more symptoms of disorders associated with bronchoconstriction which comprises:
(a) a quaternary ammonium muscarinic receptor antagonist in a concentration based on the ammonium of between about 0.0005% and about 5% by weight;
(b) a pharmacologically acceptable fluid; and
(c) a pharmacologically acceptable preservative,
wherein the pH of the preparation is adjusted between about 2.0 to about 4.5 with an acid and the quaternary ammonium muscarinic receptor antagonist is dissolved in the fluid and optionally includes a pharmacologically acceptable complexing agent, stabilizer, a pharmacologically acceptable cosolvent, or other pharmacologically acceptable adjuvants and additives; and
(iii) packaging material which includes instructions for the administration of the nebulizable composition to a patient in need of treatment or amelioration of one or more symptoms of disorders associated with bronchoconstriction;
wherein the nebulizer releases the nebulized composition upon inhalation by the patient and ceases release of the nebulized composition when inhalation is stopped and the administration of the nebulizable composition by the nebulizer results in minimal exposure of the nebulized composition to the body surface of the patient.

2. The kit of claim 1, wherein the body surface of the patient is the face and eyes.

3. The kit of claim 1, wherein the nebulizer is a breath actuated nebulizer.

4. The kit of claim 3, wherein the quaternary ammonium muscarinic receptor antagonist is an ipratropium or tiotropium compound.

5. The kit of claim 4, wherein the tiotropium compound is tiotropium bromide.

6. The kit of claim 5, wherein the nebulizable composition further comprises an additional compound for the treatment of bronchoconstriction which is selected from the group consisting of a β₂-adrenoreceptor agonist, a dopamine (D₂) receptor agonist, a steroidal anti-inflammatory agent, an anticholinergic agent, an IL-5 inhibitor, an antisense modulator of IL-5, a tryptase inhibitor, a leukotriene receptor antagonist, a 5-lapoxygenase inhibitor, an anti-IgE antibody, an antihistamine, an anti-allergic agent and mixtures thereof.

7. The kit of claim 5, wherein the nebulizable composition is contained in a unit dose vial.

8. The kit of claim 5, wherein the instructions recite once a day prior to going to sleep administration of the nebulizable composition.

9. A method of treatment, prevention or amelioration or one or more symptoms of diseases or disorders associated with bronchoconstriction which comprises of delivering the nebulizable composition via the nebulizer from the kit of claim 1, wherein the administration of the nebulizable composition by the nebulizer results in minimal exposure of the nebulized composition to the body surface of the patient.

10. The method of claim 9, wherein the body surface of the patient is the face and eyes.

11. The method of claim 10, wherein the loss of quaternary ammonium muscarinic receptor antagonists delivered to the mouth and lungs of the patient is less than 0.001% w/w.

12. The method of claim 11, wherein the nebulizer allows for the release of the nebulized composition upon inhalation by the patient and ceases release of the nebulized composition when inhalation has ended.

13. The method of claim 12, wherein the nebulizer is a breath actuated nebulizer.

14. The method of claim 13, wherein the quaternary ammonium muscarinic receptor antagonist is an ipratropium or tiotropium compound.

15. The method of claim 14, wherein the tiotropium compound is tiotropium bromide.

16. The method of claim 15, wherein the nebulizable composition further comprises an additional compound for the treatment of bronchostriction which is selected from the group consisting of a β₂-adrenoreceptor agonist, a dopamine (D₂) receptor agonist, a steroidal anti-inflammatory agent, an anticholinergic agent, an IL-5 inhibitor, an antisense modulator of IL-5, a tryptase inhibitor, a leukotriene receptor antagonist, a 5-lapoxygenase inhibitor, an anti-IgE antibody, an antihistamine, an anti-allergic agent and mixtures thereof.

17. The method of claim 15, wherein the nebulizable composition is contained in a unit dose vial.

18. The method of claim 15, wherein the nebulizable composition are administered once a day administration prior to the patient going to sleep.
